(19) United States
(12) Patent Application Publication
(10) Pub. No.: US 2023/0107598 A1
(43) Pub. Date: Apr. 6, 2023

(54) SORGHUM DOUBLED HAPLOID PRODUCTION SYSTEM

(71) Applicant: PIONEER HI-BRED INTERNATIONAL, INC., JOHNSTON, IA (US)

(72) Inventors: CLEVE DOUGLAS FRANKS, CANYON, TX (US); TANVEER HUSSAIN, URBANDALE, IA (US)

(73) Assignee: PIONEER HI-BRED INTERNATIONAL, INC., JOHNSTON, IA (US)

(21) Appl. No.: 17/904,983

(22) PCT Filed: Feb. 23, 2021
(86) PCT No.: PCT/US2021/019195

§ 371 Date: Aug. 25, 2022

Related U.S. Application Data
(60) Provisional application No. 62/983,381, filed on Feb. 28, 2020.

(51) Int. Cl. A01H 1/02; A01H 6/46

(52) U.S. Cl. CPC A01H 1/021; A01H 6/4666

(57) ABSTRACT

Methods and compositions useful in sorghum breeding are provided. Methods of producing haploid sorghum embryos, plants, seeds, and plant parts are provided. The sorghum haploid embryos produced may be doubled to produce doubled haploid embryos, plants, seeds, and plant parts as part of a sorghum breeding program.

### FIELD

The present disclosure relates to the field of sorghum breeding, including haploid and doubled haploid production of sorghum plants.

### BACKGROUND

Sorghum, for example Sorghum bicolor L., (2n=2x=20), is an important and valuable food and feed grain crop. In addition, its vegetative parts are used for forage, syrup and shelter. Thus, a continuing goal of plant breeders is to develop stable high yielding sorghum hybrids that are agronomically sound. The reasons for this goal are to maximize the amount of grain produced on the land used and to supply food for both animals and humans.

However, the development of new inbred and hybrid sorghum plants is slow and costly, lagging behind that of other crops such as maize. One reason is that homozygous plants are basic for product development and commercialization. To obtain selectable homozygous plants in a breeding program requires several generations of self-pollination and segregation analysis. This is an inefficient use of labor and time resources. It would therefore be useful to develop a method to reduce hand pollination steps normally required to obtain a homozygous sorghum plant and to reduce the amount of time required to obtain a homozygous population of sorghum plants. One way to obtain homozygous sorghum plants without the need to self-pollinated multiple generations is to produce sorghum haploids and then double the chromosomes to form doubled sorghum haploids, thereby positively impacting genetic gain. Haploid induction and subsequent genome doubling are the two main steps required for the double haploid (DH) technology. In some plant species, inefficient haploid genome doubling due to lack of available technology is considered a major obstacle for implementation of DH technology in commercial breeding programs and thereby increase genetic gain. The biological mechanisms underlying selective chromosome elimination resulting from interspecific pollinations are not well understood.

The present disclosure provides methods and compositions useful in sorghum breeding. The present disclosure provides methods and compositions to produce haploid sorghum embryos, efficient doubling and generation of double haploid sorghum plants.

### SUMMARY

The present disclosure comprises methods and compositions useful in sorghum breeding. In an aspect, a method of producing a sorghum haploid embryo comprising pollinating a female sorghum diploid plant with pollen from a Pennisetum plant and obtaining the sorghum haploid embryo is provided. In an aspect, the female sorghum diploid plant is an F1, F2, F3, or a backcross derived sorghum plant. In an aspect, the F1, F2, F3, or the backcross derived sorghum plant is a Sorghum bicolor F1, F2, F3, or backcross derived sorghum plant. In an aspect, the sorghum plant is emasculated. In an aspect, the Pennisetum plant is selected from the group consisting of Pennisetum glaucum, Pennisetum cenchroides, Pennisetum americanum (L.), Pennisetum typhoides auct., Cenchrus ciliaris L, and Panicum sumatrense. In an aspect, the Pennisetum plant is Pennisetum glaucum. In an aspect, the method further comprises selecting the sorghum haploid embryo. In an aspect, the selecting comprises using a flow cytometer test. In an aspect, the method further comprises generating a sorghum haploid seedling from the sorghum haploid embryo. In an aspect, the method further comprises selecting the sorghum haploid seedling. In an aspect, the selecting comprises using a flow cytometer test. In an aspect, a sorghum haploid plant is produced by growing the sorghum haploid embryo or the sorghum haploid seedling produced by the methods disclosed herein for a sufficient time to produce the sorghum haploid plant. In an aspect, the method further comprises exposing the sorghum haploid embryo to a chromosome doubling agent. In an aspect, the method further comprises exposing the sorghum haploid plant to a chromosome doubling agent.

In an aspect, a method of producing a sorghum haploid embryo comprising pollinating a female sorghum diploid plant with pollen from a pearl millet diploid plant or a diploid plant of a close genetic relative of pearl millet and obtaining the sorghum haploid embryo is provided. In an aspect, the female sorghum diploid plant is an F1, F2, F3, or a backcross derived sorghum plant. In an aspect, the F1, F2, F3, or the backcross derived sorghum plant is a Sorghum bicolor F1, F2, F3, or backcross derived sorghum plant. In an aspect, the sorghum plant is emasculated. In an aspect, the pearl millet diploid plant or the diploid plant of a close genetic relative of pearl millet is selected from the group consisting of Pennisetum glaucum, Pennisetum americanum (L.), Pennisetum cenchroides, Panicum americanum L, Cenchrus ciliaris L, Setaria viridis, Eleusine coracana, Panicum sumatrense, Cenchrus americanus (L.), Pennisetum typhoides auct., Setaria glauca (L.), and Setaria lutescens. In an aspect, the pearl millet diploid plant or the diploid plant of a close genetic relative of pearl millet is Pennisetum glaucum. In an aspect, the method further comprises selecting the sorghum haploid embryo. In an aspect, the selecting comprises using a flow cytometer test. In an aspect, the method further comprises generating a sorghum haploid seedling from the sorghum haploid embryo. In an aspect, the method further comprises selecting the sorghum haploid seedling. In an aspect, the selecting comprises using a flow cytometer test. In an aspect, a sorghum haploid plant is produced by growing the sorghum haploid embryo or the sorghum haploid seedling produced by the methods disclosed herein for a sufficient time to produce the sorghum haploid plant. In an aspect, the method further comprises exposing the sorghum haploid embryo to a chromosome doubling agent. In an aspect, the method further comprises exposing the sorghum haploid plant to a chromosome doubling agent. In an aspect, a method of producing a sorghum doubled haploid embryo, a sorghum doubled haploid seedling, or a sorghum doubled haploid plant is provided by contacting the sorghum haploid embryo or the sorghum haploid seedling or the sorghum haploid plant produced by the methods disclosed herein with a chromosome doubling agent and thereby producing the doubled haploid sorghum embryo, the doubled haploid sorghum seedling, or the doubled haploid sorghum plant. In an aspect, the chromosome doubling agent is selected from the group consisting of colchicine, pronamide, dithipyr, oryzalin, AMP, and trifluralin.

In an aspect, a plant, non-seed plant part, seed, or cell of a pearl millet variety or a variety of a close genetic relative of pearl millet capable of inducing a sorghum haploid embryo when used to pollinate a sorghum variety, wherein the pearl millet variety or the variety of a close genetic relative of pearl millet comprises a heterologous polynucleotide or an introduced genetic modification is provided. In an aspect, a sorghum haploid embryo or a sorghum haploid seedling is produced by pollinating an emasculated sorghum spikelet with pollen of the pearl millet variety or the variety of a close genetic relative of pearl millet of that comprises a heterologous polynucleotide or an introduced genetic modification. In an aspect, a sorghum haploid plant is produced by growing the sorghum haploid embryo or the sorghum haploid seedling produced by pollinating an emasculated sorghum spikelet with pollen of the pearl millet variety or the variety of a close genetic relative of pearl millet of that comprises a heterologous polynucleotide or an introduced genetic modification for a sufficient time to produce the sorghum haploid plant.

In an aspect, a method of producing a sorghum doubled haploid embryo comprising contacting a sorghum female reproductive system or a component thereof with a pearl millet or a genetically related species of pearl millet male reproductive system or a component thereof to produce a haploid sorghum cell; contacting the haploid sorghum cell with a chromosome doubling agent; and producing the sorghum doubled haploid embryo from the haploid sorghum cell is provided. In an aspect, the sorghum doubled haploid embryo is further grown to a sorghum doubled haploid plant. In an aspect, the sorghum female reproductive system or a component thereof is from an F1, F2, F3, or a backcross derived sorghum plant. In an aspect, the F1, F2, F3, or the backcross derived sorghum plant is a Sorghum bicolor F1, F2, F3, or backcross derived sorghum plant. In an aspect, the pearl millet or the genetically related species of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens. In an aspect, the chromosome doubling agent is selected from the group consisting of colchicine, pronamide, dithipyr, oryzalin, AMP, and trifluralin.

In an aspect, a method of obtaining a doubled haploid sorghum plant comprising: (a) pollinating an emasculated sorghum diploid spikelet with pollen of a diploid pearl millet plant or a diploid plant of a close genetic relative of pearl millet to produce at least one haploid sorghum embryo or at least one haploid sorghum seedling; (b) applying a growth regulator to the at least one haploid sorghum embryo or the at least one haploid sorghum seedling; (c) isolating the at least one haploid sorghum embryo from caryopsis between 10-20 days after step (a); (d) obtaining the at least one haploid sorghum seedling between 3-4 weeks after step (b); (e) confirming the ploidy level of the isolated haploid sorghum embryo of (c) or the isolated haploid sorghum seedling of (d); (f) contacting the haploid sorghum embryo of (c) or the haploid sorghum seedling of (d) with a chromosome doubling agent to produce at least one doubled haploid sorghum embryo cell or at least one doubled haploid sorghum seedling cell; (g) culturing the doubled sorghum embryo cell or the doubled sorghum seedling cell on a germination medium; and (h) generating a doubled haploid sorghum plant from the doubled haploid sorghum embryo cell or the doubled haploid sorghum seedling cell is provided. In an aspect, the emasculated sorghum diploid spikelet is an anther emasculated sorghum diploid spikelet. In an aspect, the haploid sorghum embryo of step (b) is 0.1 mm to 3 mm in length. In an aspect, the at least one haploid sorghum embryo of step (c) is 0.1 mm to 3 mm in length and is isolated from caryopsis between 14-17 days after step (a). In an aspect, the chromosome doubling agent comprises an anti-microtubule agent. In an aspect, the chromosome doubling agent is selected from the group consisting of colchicine, pronamide, dithipyr, oryzalin, AMP, and trifluralin. In an aspect, in step (f) the haploid sorghum embryo or haploid sorghum seedling is in contact with the chromosome doubling agent for 3 hours to 48 hours. In an aspect, the diploid pearl millet plant or the diploid plant of a close genetic relative of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens. In an aspect, the sorghum diploid spikelet is from an F1, F2, F3, or a backcross derived sorghum plant. In an aspect, the F1, F2, F3, or the backcross derived sorghum plant is a Sorghum bicolor F1, F2, F3, or backcross derived sorghum plant.

In an aspect, a method of transforming a cell of an isolated sorghum haploid embryo or an isolated sorghum haploid seedling with a polynucleotide of interest using a bacterial-mediated transformation and treating the transformed cell with a chromosome doubling agent to produce a transgenic homozygous sorghum plant cell, wherein the cell of the isolated sorghum haploid embryo or the cell of the isolated sorghum haploid seedling is produced by pollinating an emasculated sorghum diploid spikelet with pollen of a diploid pearl millet plant or a diploid plant of a close genetic relative of pearl millet is provided. In an aspect, the emasculated sorghum diploid spikelet is an anther emasculated sorghum diploid spikelet. In an aspect, a morphogenic gene has been introduced into the emasculated sorghum diploid spikelet or the pollen of the diploid pearl millet plant or the diploid plant of a close genetic relative of pearl millet by stable or transient transformation. In an aspect, the morphogenic gene improves transformation frequency or regeneration frequency. In an aspect, the method further comprising regenerating a transgenic homozygous sorghum plant from the transgenic homozygous sorghum plant cell. In an aspect, the diploid pearl millet plant or the diploid plant of a close genetic relative of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens. In an aspect, the sorghum diploid spikelet is from an F1, F2, F3, or a backcross derived sorghum plant. In an aspect, the F1, F2, F3, or the backcross derived sorghum plant is a Sorghum bicolor F1, F2, F3, or backcross derived sorghum plant. In an aspect, the chromosome doubling agent comprises an anti-microtubule agent. In an aspect, the chromosome doubling agent is selected from the group consisting of colchicine, pronamide, dithipyr, oryzalin, AMP, and trifluralin. In an aspect, the transformed cell is treated with the chromosome doubling agent for 3 hours to 48 hours.

In an aspect, a method of editing a sorghum plant genome, comprising: a) obtaining a non-sorghum plant, wherein the non-sorghum plant is capable of haploid induction in a sorghum plant, and wherein the non-sorghum plant expresses a DNA modification enzyme and optionally at least one guide nucleic acid, in its male reproductive system; b) obtaining a sorghum plant comprising a target genome for site-directed genetic modification; c) pollinating the sorghum plant with pollen from the non-sorghum plant; and d) selecting at least one haploid sorghum progeny produced by the pollination of step (c) wherein, the haploid sorghum progeny comprises the genome of the sorghum plant but not the genome of the non-sorghum plant, and wherein the genome of the sorghum haploid progeny has been modified by the DNA modification enzyme delivered by the non-sorghum plant, providing an edited haploid sorghum progeny is provided. In an aspect, the sorghum plant is pearl millet or a close genetic relative of pearl millet. In an aspect, the pearl millet or the close genetic relative of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens. In an aspect, the DNA modification enzyme is a site-directed nuclease selected from the group consisting of meganucleases, zinc-finger nucleases, transcription-activator like effector nucleases (TALENs), Cas nuclease fusions, Cas9 nuclease, Cas12f, Cfp1 nuclease, dCas9-FokI, dCpf1-FokI, chimeric Cas-cytidine deaminase, chimeric Cas-adenine deaminase, chimeric FEN1-FokI, and Mega-TALs, a nickase Cas9 (nCas9), nCas9-deaminase fusion, chimeric dCas9 non-FokI nuclease and dCpf1 non-FokI nuclease. In an aspect, the pollinating step comprises transferring pollen or a pollen-derived component from the non-sorghum plant. In an aspect, the optionally at least one guide nucleic acid is a guide RNA. In an aspect, the edited haploid sorghum progeny is treated with a chromosome doubling agent, thereby creating an edited doubled haploid sorghum plant. In an aspect, the chromosome doubling agent is colchicine, pronamide, dithipyr, trifluralin, or another anti-microtubule agent. In an aspect, the non-sorghum plant is a transgenic pearl millet plant or a transgenic close genetic relative of pearl millet. In an aspect, the transgenic pearl millet or the transgenic close genetic relative of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens. In an aspect, the editing results in an improved agronomic trait. In an aspect, the improved agronomic trait is selected from the group consisting of improved disease resistance, improved drought tolerance, improved heat tolerance, improved cold tolerance, improved salinity tolerance, improved metal tolerance, improved herbicide tolerance, improved water use efficiency, improved nitrogen utilization, improved nitrogen fixation, improved pest resistance, improved herbivore resistance, improved pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, improved modulation of a metabolite, improved modulation of the proteome, increased seed weight, altered seed carbohydrate composition, altered seed oil composition, altered seed protein composition, altered seed nutrient composition, as compared to an isoline plant not comprising a modification. In an aspect, the editing results in tolerance or resistance to one or more herbicides. In an aspect, the one or more herbicides is selected from the group consisting of imidazolinone, ACCase Inhibitors (including Aryloxyphenoxypropionate (FOPs), cyclohexanedione (DIMs), and phenylpyrazolin (DENs)), glufosinate, and PPO inhibitor herbicides. In an aspect, the editing results in tolerance or resistance to one or more biological pests.

### DETAILED DESCRIPTION

Haploids are generated through the culture of immature male and female gametophytes, and through inter- and intra-specific via chromosome elimination as described herein. Sorghum haploids are obtained from the progeny of crosses between a sorghum plant and another plant from different species by a process of selected chromosome elimination. In an embodiment, the non-sorghum plant is pearl millet.

In the description and examples that follow, a number of terms are used herein. Terms used in the claims and specification are defined as set forth below unless otherwise specified. In order to provide a clear and consistent understanding of the specification and claims, including the scope to be given such terms, the following descriptions are provided:

“Chromosome doubling” generally refers to that each of the chromosomes in a cell is duplicated resulting in a doubling of the number of chromosomes in the cell.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; U.S. Pat. No. 9,879,269). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example U.S. Pat. Pub. No. US20150059010A1), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “polynucleotide-guided endonuclease”, and “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems. In some aspects, the guide polynucleotide/Cas endonuclease complex is provided as a ribonucleoprotein (RNP), wherein the Cas endonuclease component is provided as a protein and the guide polynucleotide component is provided as a ribonucleotide.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In some aspects, a “polynucleotide modification template” is provided that comprises at least one nucleotide modification when compared to the nucleotide sequence to be edited. A nucleotide modification can be at least one nucleotide substitution, addition, deletion, or chemical alteration. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In some aspects, a polynucleotide of interest is inserted at a target site and provided as part of a “donor DNA” molecule. As used herein, “donor DNA” is a DNA construct that comprises a polynucleotide of interest to be inserted into the target site. In some aspects, the donor DNA construct may further comprise a first and a second region of homology that flank the polynucleotide of interest. The first and second regions of homology of the donor DNA share homology to a first and a second genomic region, respectively, present in or flanking the target site of the cell or organism genome. The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963). The amount of homology or sequence identity shared by a target and a donor polynucleotide can vary and includes total lengths and/or regions.

“Callus” refers to a dedifferentiated proliferating mass of cells or tissue.

The phrases “contacting”, “comes in contact with” or “placed in contact with” can be used to mean “direct contact” or “indirect contact”. For example, the medium comprising a chromosome doubling agent may have direct contact with the haploid cell or the medium comprising the chromosome doubling agent may be separated from the haploid cell by filter paper, plant tissues, or other cells thus the chromosome doubling agent is transferred through the filter paper or cells to the haploid cell.

A haploid plant has a single set (genome) of chromosomes and the reduced number of chromosomes (n) in the haploid plant is equal to that in the gamete (for Sorghum bicolor, n=10).

A diploid plant has two sets (genomes) of chromosomes and the chromosome number (2n) is equal to that in the zygote (for Sorghum bicolor, 2n=20).

A doubled haploid or doubled haploid plant or cell is one that is developed by the doubling of a haploid set of chromosomes. A plant or seed that is obtained from a doubled haploid plant that is selfed any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes. For example, a plant will be considered a doubled haploid plant if it contains viable gametes, even if it is chimeric.

A “haploid immature embryo” generally refers to an embryo formed after one sperm nucleus from a pollen grain fuses with the polar nuclei in the embryo sac to create a triploid (3N) endosperm and before dry down or an embryo resulting from a haploid induction after wide-cross or wide-hybridization with a different species.

A “doubled haploid embryo” is an embryo that has one or more cells that contain 2 sets of homozygous chromosomes.

“Effective amount” generally refers to an amount of an agent, compound or plant growth regulator that is capable of causing the desired effect on an organism. It is recognized that an “effective amount” may vary depending on factors, such as, for example, the organism, the target tissue of the organism, the method of administration, temperature, light, relative humidity and the like. Further, it is recognized that an “effective amount” of a particular agent may be determined by administering a range of amounts of the agent to an organism and then determining which amount or amounts cause the desired effect.

As used herein, “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance.

As used herein, “improved agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions disclosed herein.

As used herein, the term “embryogenesis factor” means a gene that when expressed enhances cellular reprogramming that can promote improved formation of a somatically-derived structure. When embryogenesis factors are co-expressed with a morphogenic gene improved methods for obtaining a plant are provided. More precisely, ectopic expression of an embryogenesis factor stimulates de novo formation of an organogenic structure, for example a structure from embryogenic callus tissue, that can improve the formation of an embryo. This stimulated de novo embryogenic formation occurs either in the cell in which the embryogenesis factor is expressed, or in a neighboring cell. An embryogenesis factor gene can be a transcription factor that regulates expression of other genes, or a gene that influences hormone levels in a plant cell, or a gene that influences an enzyme affecting cellular reprogramming in a plant cell, any of which can stimulate embryogenic changes. As used herein, the term “embryogenesis factor” means an embryogenesis factor gene and/or the protein expressed by an embryogenesis factor gene acting as a cellular reprogramming agent.

An embryogenesis factor is involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. An embryogenesis factor can be used in combination with a morphogenic gene to improve cellular reprogramming involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or combinations thereof. Embryogenesis factors include, but are not limited to, polynucleotides encoding a transcription factor bHLH30-like polypeptide, a hybrid D-type cyclin polypeptide, a mitogen-activated kinase protein polypeptide, a plant lipid transfer polypeptide, a cyclin delta-2 polypeptide. an oberon-like protein-like polypeptide, a polynucleotide adenylyltransferase polypeptide, a GATA zinc finger polypeptide, a homeobox-leucine zipper polypeptide, a hydrolase polypeptide, a telomerase reverse transcriptase polypeptide, a zinc finger polypeptide, a GRAS family transcription factor polypeptide, a mlo defense gene homolog polypeptide, a 3-ketoacyl-CoA synthase 11-like polypeptide, a phytosulfokine polypeptide; and combinations comprising any two or more embryogenesis factor polypeptides.

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. A morphogenic gene may be stably incorporated into the genome of a plant or it may be transiently expressed. Morphogenic genes involved in plant metabolism, organ development, stem cell development, cell growth stimulation, organogenesis, embryogenesis, regeneration, somatic embryogenesis initiation, accelerated somatic embryo maturation, initiation and/or development of the apical meristem, initiation and/or development of shoot meristem, initiation and/or development of shoots, or a combination thereof, such as WUS/WOX genes (WUS1, WUS2, WUS3, WOX2A, WOX4, WOX5, or WOX9) see U.S. Pat. Nos. 7,348,468 and 7,256,322 and U.S. Pat. Pub. No. US20170121722A1; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Other morphogenic genes of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

Morphogenic genes useful in the present disclosure include, but are not limited to, those disclosed in WO2019060383, published on Mar. 28, 2019. Other morphogenic genes useful in the present disclosure include, but are not limited to, LEC1 (Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabiopsis AGL15 gene (Harding et al., 2003, Plant Physiol. 133:653-663).

As used herein, the term “transcription factor” means a protein that controls the rate of transcription of specific genes by binding to the DNA sequence of the promoter and either up-regulating or down-regulating expression. Examples of transcription factors, which are also morphogenic genes, include members of the AP2/EREBP family (including the BBM (ODP2), plethora and aintegumenta sub-families, CAAT-box binding proteins such as LEC1 and HAP3, and members of the MYB, bHLH, NAC, MADS, bZIP and WRKY families.

The term “medium” includes compounds in liquid, gas, or solid state.

“Organogenesis” generally refers to the developmental process wherein a cell or group of cells gives rise to an organ such as, for example, a shoot, a bud, or a root.

As used herein, the term “plant” includes reference to whole plants, plant organs (e.g., leaves, stems, roots, etc.), seeds and plant cells and progeny of same. “Plant cell”, as used herein includes, without limitation, seeds, suspension cultures, embryos, meristematic regions, callus tissue, leaves, roots, shoots, gametophytes, sporophytes, pollen, and microspores.

Pearl millet belongs to genus: Pennisetum and species: glaucum of the family: Poaceae. Pearl millet (Pennisetum glaucum) is the most widely grown type of millet. Close genetic relatives of pearl millet or species genetically related to or similar to pearl millet or millet include members of the family Poaceae for example including but not limited to, Setariopsis glauca, Setaria sericea, Setaria viridis (foxtail millet) millet, Eleusine coracana (finger millet), Penicillaria willdenowii, Penicillaria typhoidea, Panicum sericeum, Panicum sumatrense (known as little millet), Panicum lutescens, Panicum involucratum, Panicum indicum, Panicum hokoides, Panicum glaucum, Panicum compressum, Panicum americanum L., and Pennisetum including but not limited to Pennisetum typhoideum var. plukenetii, Pennisetum typhoideum var. echinurus, Pennisetum typhoideum, Pennisetum typhoides, Pennisetum spicatum subsp. willdenowi, Pennisetum spicatum var. typhoideum, Pennisetum spicatum var. macrostachyum, Pennisetum spicatum var. longipedunculatum, Pennisetum spicatum var. echinurus, Pennisetum spicatum (L.), Pennisetum solitarium, Pennisetum pycnostachyum, Pennisetum plukenetii, Pennisetum nigritarum var. macrostachyum, Pennisetum nigritarum var. deflexum, Pennisetum nigritarum, Pennisetum megastachyum, Pennisetum malacochaete, Pennisetum maiwa, Pennisetum linnaei, Pennisetum leonis, Pennisetum indicum, Pennisetum giganteum, Pennisetum gibbosum, Pennisetum gambiense, Pennisetum echinurus, Pennisetum cinereum, Pennisetum cereal, Pennisetum aureum, Pennisetum ancylochaete, Pennisetum americanum (L.), Pennisetum americanum subsp. typhoideum, Pennisetum americanum subsp. spicatum, Pennisetum americanum f. echinurus, Pennisetum americanum, Pennisetum albicauda.

“Ploidy” generally refers to the number of complete sets of chromosomes in the nucleus of a cell. A “haploid” cell has one set of chromosomes, and a “diploid” cell has two sets.

“Progeny” generally refers to descendents of a particular cell or plant which may comprise at least a portion of the transgene inserted at the locus of the genome of the TO plant cell, in case of genome modifications. For example, progeny can be seeds developed on a plant and plants derived from such seeds. For example, progeny of a plant include seeds formed on doubled haploid (DH) plants, genetically modified T0, T1, T2 and subsequent generation plants, and plants derived from such seeds.

As used herein “recombinant” means a cell or vector, that has been modified by the introduction of a heterologous nucleic acid or a cell derived from a cell so modified. Thus, for example, a recombinant cell is a cell expressing a gene that is not found in identical form or location within the native (non-recombinant) cell or a cell that expresses a native gene in an expression pattern that is different from that of the native (non-recombinant) cell for example, the native gene is abnormally expressed, over expressed, under expressed, has reduced expression or is not expressed at all because of deliberate human intervention. The term “recombinant” as used herein does not encompass the alteration of a cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

“Somatic embryo” generally refers to an embryo that develops from a somatic cell. The developmental process by which a somatic embryo develops from a cell is known as “somatic embryogenesis.” Such a “somatic embryo” is distinct from a “zygotic embryo” which develops from a zygote.

A sorghum plant generally refers to a plant of the genus Sorghum, in particular of the species Sorghum bicolor, Sorghum halepense, Sorghum sudanense, Sorghum arundinaceum and/or Sorghum propinquum or their hybrids and all varieties derived from them.

As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic. A transgenic plant is defined as a mature, fertile plant that contains a transgene.

As used herein, the term “transgene” refers to a gene that has been transferred naturally, or by any of a number of genetic engineering techniques from one organism to another.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The methods provided herein rely upon the use of bacteria-mediated and/or biolistic-mediated gene transfer to produce regenerable plant cells having an incorporated nucleotide sequence of interest. Bacterial strains useful in the methods of the disclosure include, but are not limited to, a disarmed Agrobacteria including, but are not limited to, AGL-1, EHA105, GV3101, LBA4404, LBA4404 THY-, and LBA4404 THY-Tn904-, an Ochrobactrum bacteria (see U.S. Pat. Pub. No. US20180216123A1) or a Rhizobiaceae bacteria (see U.S. Pat. No. 9,365,859).

A highly efficient microprojectile transformation system for sorghum using immature embryos (IEs) of inbred line Tx430 was described in Liu and Godwin (2012) Plant Cell Rep. 2012 June; 31(6): 999-1007, incorporated herein by reference.

In some aspects, methods and compositions are provided for modifying naturally-occurring polynucleotides or integrated transgenic sequences, including regulatory elements, coding sequences, and non-coding sequences. These methods and compositions are also useful in targeting nucleic acids to pre-engineered target recognition sequences in the genome. Modification of polynucleotides may be accomplished, for example, by introducing single- or double-strand breaks into the DNA molecule.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.)), meganucleases (see e.g., U.S. Pat. No. 8,338,157; Gao et al. (2010) Plant Journal 1:176-187), TAL effector nucleases or TALENs (see e.g., U.S. Pat. Pub. No. US20110145940A1, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. U.S. Pat. No. 9,879,269).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The HDR pathway is another cellular mechanism to repair double-stranded DNA breaks and includes homologous recombination (HR) and single-strand annealing (SSA) (Lieber. 2010 Annu. Rev. Biochem. 79:181-211).

### CRISPR-Cas Methods for Gene Editing

Examples of a Cas endonuclease include but are not limited to Cas9 and Cpf1. Cas9 (formerly referred to as Cas5, Csn1, or Csx12) is a Class 2 Type II Cas endonuclease (Makarova et al. 2015, Nature Reviews Microbiology Vol. 13:1-15). A Cas9-gRNA complex recognizes a 3′ PAM sequence (NGG for the S. pyogenes Cas9) at the target site, permitting the spacer of the guide RNA to invade the double-stranded DNA target, and, if sufficient homology between the spacer and protospacer exists, generate a double-strand break cleavage. Cas9 endonucleases comprise RuvC and HNH domains that together produce double strand breaks, and separately can produce single strand breaks. For the S. pyogenes Cas9 endonuclease, the double-strand break leaves a blunt end. Cpf1 is a Class 2 Type V Cas endonuclease, and comprises nuclease RuvC domain but lacks an HNH domain (Yamane et al., 2016, Cell 165:949-962). Cpf1 endonucleases create “sticky” overhang ends.

Some uses for Cas9-gRNA systems at a genomic target site include but are not limited to insertions, deletions, substitutions, or modifications of one or more nucleotides at the target site; modifying or replacing nucleotide sequences of interest (such as a regulatory elements); insertion of polynucleotides of interest; gene knock-out; gene-knock in; modification of splicing sites and/or introducing alternate splicing sites; modifications of nucleotide sequences encoding a protein of interest; amino acid and/or protein fusions; and gene silencing by expressing an inverted repeat into a gene of interest.

The process for editing a genomic sequence at a Cas9-gRNA double-strand-break site with a modification template generally comprises: providing a host cell with a Cas9-gRNA complex that recognizes a target sequence in the genome of the host cell and is able to induce a double-strand-break in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the double-strand break.

To facilitate optimal expression and nuclear localization for eukaryotic cells, the gene comprising the Cas endonuclease may be optimized as described in U.S. Pat. Pub. No. US20180258417A1, and then delivered into cells as DNA expression cassettes. In some aspects, the Cas endonuclease is provided as a polypeptide. In some aspects, the Cas endonuclease is provided as a polynucleotide encoding a polypeptide. In some aspects, the guide RNA is provided as a DNA molecule encoding one or more RNA molecules. In some aspects, the guide RNA is provided as RNA or chemically-modified RNA. In some aspects, the Cas endonuclease protein and guide RNA are provided as a ribonucleoprotein complex (RNP).

A catalytically active or inactive Cas protein, described herein, can also be in fusion with a molecule that directs editing of single or multiple bases in a polynucleotide sequence, for example a site-specific deaminase that can change the identity of a nucleotide, for example from C⋅G to T⋅A or an A⋅T to G⋅C (Gaudelli et al., Programmable base editing of A⋅T to G⋅C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A base editing fusion protein may comprise, for example, an active (double strand break creating), partially active (nickase) or deactivated (catalytically inactive) Cas endonuclease and a deaminase (such as, but not limited to, a cytidine deaminase, an adenine deaminase, APOBEC1, APOBEC3A, BE2, BE3, BE4, ABEs, or the like). Base edit repair inhibitors and glycosylase inhibitors (e.g., uracil glycosylase inhibitor (to prevent uracil removal)) are contemplated as other components of a base editing system, in some embodiments.

### Zinc Finger Mediated Genome Editing

As an example, the genetically modified cell or plant described herein, is generated using a zinc finger nuclease-mediated genome editing process. The process for editing a chromosomal sequence includes for example: (a) introducing into a cell at least one nucleic acid encoding a zinc finger nuclease that recognizes a target sequence in the chromosomal sequence and is able to cleave a site in the chromosomal sequence, and, optionally, (i) at least one donor polynucleotide that includes a sequence for integration flanked by an upstream sequence and a downstream sequence that exhibit substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. A zinc finger nuclease includes a DNA binding domain (i.e., zinc finger) and a cleavage domain (i.e., nuclease). The nucleic acid encoding a zinc finger nuclease may include DNA or RNA. Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Choo et al. (2000) Curr. Opin. Struct. Biol. 10:411-416; and Doyon et al. (2008) Nat. Biotechnol. 26:702-708; Santiago et al. (2008) Proc. Natl. Acad. Sci. USA 105:5809-5814; Urnov, et al., (2010) Nat Rev Genet. 11(9):636-46; and Shukla, et al., (2009) Nature 459 (7245):437-41. An engineered zinc finger binding domain may have a novel binding specificity compared to a naturally-occurring zinc finger protein. As an example, the algorithm of described in U.S. Pat. No. 6,453,242 may be used to design a zinc finger binding domain to target a preselected sequence. Nondegenerate recognition code tables may also be used to design a zinc finger binding domain to target a specific sequence (Sera et al. (2002) Biochemistry 41:7074-7081). Tools for identifying potential target sites in DNA sequences and designing zinc finger binding domains may be used (Mandell et al. (2006) Nuc. Acid Res. 34:W516-W523; Sander et al. (2007) Nuc. Acid Res. 35:W599-W605).

An exemplary zinc finger DNA binding domain recognizes and binds a sequence having at least about 80% sequence identity with the desired target sequence. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

A zinc finger nuclease also includes a cleavage domain. The cleavage domain portion of the zinc finger nucleases may be obtained from any endonuclease or exonuclease. Non-limiting examples of endonucleases from which a cleavage domain may be derived include, but are not limited to, restriction endonucleases and homing endonucleases. See, for example, 2010-2011 Catalog, New England Biolabs, Beverly, Mass.; and Belfort et al. (1997) Nucleic Acids Res. 25:3379-3388. Additional enzymes that cleave DNA are known (e.g., 51 Nuclease; mung bean nuclease; pancreatic DNase I; micrococcal nuclease; yeast HO endonuclease). One or more of these enzymes (or functional fragments thereof) may be used as a source of cleavage domains.

### Meganuclease-Based Genome Editing

Another example for genetically modifying the cell or plant described herein, is by using “custom” meganucleases produced to modify plant genomes (see e.g., U.S. Pat. No. 8,338,157; Gao et al. (2010) Plant Journal 1:176-187. The term “meganuclease” generally refers to a naturally-occurring homing endonuclease that binds double-stranded DNA at a recognition sequence that is greater than 12 base pairs and encompasses the corresponding intron insertion site. Naturally-occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease.

Naturally-occurring meganucleases have been used to effectively promote site-specific genome modification in plants, yeast, Drosophila, mammalian cells and mice. Engineered meganucleases such as, for example, LIG-34 meganucleases, which recognize and cut a 22 basepair DNA sequence found in the genome of Zea mays (maize) are known (see e.g., U.S. Pat. Pub. No. US20110113509A1).

### TAL Endonucleases (TALEN)

TAL (transcription activator-like) effectors from plant pathogenic Xanthomonas are important virulence factors that act as transcriptional activators in the plant cell nucleus, where they directly bind to DNA via a central domain of tandem repeats. A transcription activator-like (TAL) effector-DNA modifying enzymes (TALE or TALEN) are also used to engineer genetic changes. See e.g., U.S. Pat. Pub. No. US20110145940A1, Boch et al., (2009), Science 326(5959): 1509-12. Fusions of TAL effectors to the FokI nuclease provide TALENs that bind and cleave DNA at specific locations. Target specificity is determined by developing customized amino acid repeats in the TAL effectors.

### Production of Sorghum Haploid Embryos

Provided are methods for 1) identifying haploid embryos at an early stage with high accuracy, 2) chromosomal doubling protocols at an early stage of embryo development, 3) plant generation from early stage embryos. The methods are generally genotype independent. The methods generally produce a high frequency of doubled haploid plants that are fertile.

One method provided comprises obtaining a doubled haploid embryo, seed, or plant by contacting a haploid embryo with a doubling agent and obtaining a doubled haploid embryo, seed, or plant.

Another method provided is obtaining a doubled haploid plant comprising the following steps: a) pollinating ovules, or stigmas, of a sorghum plant with pollen from a pearl millet plant; b) applying a growth regulator 2,4-D and thereby producing a sorghum haploid embryo or sorghum haploid seedling selecting a haploid embryo; c) contacting sorghum haploid embryo or sorghum haploid seedling with a gas, solution or solid comprising a doubling agent; and d) regenerating that embryo or seedling into a doubled haploid plant.

A method of inbred selection is provided comprising the following steps: a) cross pollinating two inbred sorghum plants; b) growing the F1 seed; c) pollinating the F1 plant with a distant species such as pearl millet to produce haploid embryos; d) contacting the haploid embryos with a chromosome doubling agent to produce doubled haploid embryos; e) generating doubled haploid plants; f) evaluating said doubled haploid plants for agronomic performance and combining ability. The development of haploids step may also be done at later generations, F2, F3, F4, etc. Producing haploids from later generations allows for additional opportunities for recombination.

The methods provided can include the use of embryo rescue. Embryo rescue is performed by contacting an embryo with a growth medium containing nutrients and generating a plant. Phytohormones may or may not be included in the embryo rescue medium.

A method of obtaining a transgenic doubled haploid embryo is provided comprising isolating a haploid embryo, transforming the haploid embryo, placing the haploid embryo on a medium comprising a chromosome doubling agent and selecting a transgenic doubled haploid embryo.

In any of these methods the chromosomes can be doubled at the immature embryo stage, at the mature seed stage, or anytime between pollination of the sorghum plant and before the germination of the sorghum haploid seed. This can also be done when a haploid seed germinates and form a haploid seedling.

Methods for obtaining homozygous sorghum plants, plant cells, and seeds are provided. Also provided are methods for obtaining sorghum haploid embryos and seeds and methods for increasing chromosomal doubling. The methods comprise contacting Sorghum haploid cells with a chromosome doubling agent. The methods also comprise pollinating a selected sorghum plant (female parent) with pollen from a pearl millet plant (male parent) to produce haploid embryos or seeds. The methods provide doubled haploid plant cells which can be generated into a plant containing homozygous genes.

The methods avoid time consuming selfing and crossing methods to obtain a homozygous trait of interest or an essentially homozygous plant. The presented methods can be used to produce doubled haploid populations that do not contain the residual heterozygosity of inbreds obtained though the traditional method of self-pollination. The methods can be useful for functional genomics, such as knock-out analysis, functional analysis of recessive genes, gene replacement, homologous recombination, gene targeting, transgene stacking, and evaluating lethal versus non-lethal analysis of genes. With the previously known diploid systems, these analyses are very complicated and costly.

Haploid cells, haploid embryos, haploid seeds, haploid seedlings or haploid plants can be treated with a chromosome doubling agent. Homozygous plants can be regenerated from haploid cells by contacting the haploid cells, such as haploid embryo cells, with chromosome doubling agents. The haploid cells may come in contact, exposed to, or treated with the doubling agent at the time of pollination, any time after pollination, typically 6 hours to 21-30 days after pollination, 6 hours to 15 days after pollination, at the mature seed stage, at the seedling stage, or at the plant stage. Suitable time for the extent of contact/exposure to the doubling agent depends on the type of doubling agent used, the concentration of the doubling agent in the growth media, cell culture conditions for the induced embryo, and may range from about 3 hours to 3 days or longer, e.g., 4, 5, 6, 7, 8, 9, 10, 11, 12, 13, 14, 15, 16, 17, 18, 19, 20 or 21 days.

Methods of chromosome doubling are disclosed in Antoine-Michard, S. et al., Plant cell, tissue organ cult., Cordrecht, the Netherlands, Kluwer Academic Publishers, 1997, 48(3):203-207; Kato, A., Maize Genetics Cooperation Newsletter 1997, 36-37; and Wan, Y. et al., TAG, 1989, 77: 889-892. Wan, Y. et al., TAG, 1991, 81: 205-211. The disclosures of which are incorporated herein by reference.

Typical methods involve contacting the cells with colchicine, anti-microtubule agents or anti-microtubule herbicides, pronamide, nitrous oxide, or any mitotic inhibitor to create homozygous doubled haploid cells. The amount of colchicine used in medium is generally 0.01%-0.2% or approximately 0.05% or APM (5-225 μM). The amount of colchicines can range from approximately 400-600 mg/L or approximately 500 mg/L. The amount of pronamide in medium is approximately 0.5-20 μM. Examples of known mitotic inhibitors are included in Table 1. Other agents may be used with the mitotic inhibitors to improve doubling efficiency. Such agents may be dimethyl sulfoxide (DMSO), adjuvants, surfactants, and the like.

TABLE 1
Chemical chromosome doubling agents

| | | |
|---|---|---|
| Common Name/Trade name | CAS | IUPAC |

Colchicine and Colchicine Derivatives

| | |
|---|---|
| colchicine/acetyltrimethylcol- | (S)-N-(5,6,7,9-tetrahydro- |
| chicinic acid | 1,2,3,10-tetramethoxy-9- |
| | oxobenzo (a) heptalen-7-yl) |
| | acetamide |
| colchicine derivatives | |

Carbamates

| | | |
|---|---|---|
| Carbetamide | (R)-1-(ethylcarbamoyl)ethyl | (2R)-N-ethyl-2- |
| | carbanilate | [[(phenylamino)carbonyl]oxy]pro- |
| | | panamide |
| chloropropham | | |
| Propham | | |

Benzamides

| | | |
|---|---|---|
| Pronamide/propyzamide | 3,5-dichloro-N-(1,1- | 3,5-dichloro-N-(1,1-dimethyl-2- |
| | dimethylpropynyl)ben- | propynyl)benzamide |
| | zamide | |
| Tebutam | | |

Benzoic Acids

| | | |
|---|---|---|
| Chlorthal dimethyl | | |
| (DCPA), | | |
| Dicamba/dianat/disugran | 3,6-dichloro-o-anisic acid | 3,6-dichloro-2-methoxybenzoic |
| (dicamba-methyl) | | acid |
| (BANVEL, CLARITY) | | |

Dinitroaniline chromosome doubling agents

| | | |
|---|---|---|
| benfluralin/benefin | N-butyl-N-ethyl-α,α,α- | N-butyl-N-ethyl-2,6-dinitro-4- |
| (BALAN) | trifluoro-2,6-dinitro-p- | (trifluoromethyl)benzenamine |
| | toluidine | |
| Butralin | (RS)-N-sec-butyl-4-tert- | 4-(1,1-dimethylethyl)-N-(1- |
| | butyl-2,6-dinitroaniline | methylpropyl)-2,6- |
| | | dinitrobenzenamine |
| Chloralin | | |
| dinitramine | N1,N1-diethyl-2,6-dinitro-4- | N3,N3-diethyl-2,4-dinitro-6- |
| | trifluoromethyl-m- | (trifluoromethyl)-1,3- |
| | phenylenediamine | benzenediamine |
| ethalfluralin (Sonalan) | N-ethyl-α,α,α-trifluoro-N-(2- | N-ethyl-N-(2-methyl-2- |
| | methylallyl)-2,6-dinitro-p- | propenyl)-2,6-dinitro-4- |
| | toluidine | (trifluoromethyl)benzenamine |
| fluchloralin | N-(2-chloroethyl)-2,6- | N-(2-chloroethyl)-2,6-dinitro-N- |
| | dinitro-N-propyl-4- | propyl-4- |
| | (trifluoromethyl)aniline | (trifluoromethyl)benzenamine |
| | or | |
| | N-(2-chloroethyl)-α,α,α- | |
| | trifluoro-2,6-dinitro-N- | |
| | propyl-p-toluidine | |
| isopropalin | 4-isopropyl-2,6-dinitro-N,N- | 4-(1-methylethyl)-2,6-dinitro- |
| | dipropylaniline | N,N-dipropylbenzenamine |
| methalpropalin | α,α,α-trifluoro-N-(2- | N-(2-methyl-2-propenyl)-2,6- |
| | methylallyl)-2,6-dinitro-N- | dinitro-N-propyl-4- |
| | propyl-p-toluidine | (trifluoromethyl)benzenamine |
| nitralin | 4-methylsulfonyl-2,6-dinitro- | 4-(methylsulfonyl)-2,6-dinitro- |
| | N,N-dipropylaniline | N,N-dipropylbenzenamine |
| oryzalin (SURFLAN) | 3,5-dinitro-N4,N4- | 4-(dipropylamino)-3,5- |
| | dipropylsulfanilamide | dinitrobenzenesulfonamide |
| pendimethalin | N-(1-ethylpropyl)-2,6- | N-(1-ethylpropyl)-3,4-dimethyl- |
| (PROWL) | dinitro-3,4-xylidine | 2,6-dinitrobenzenamine |
| prodiamine | 5-dipropylamino-α,α,α- | 2,4-dinitro-N3,N3-dipropyl-6- |
| | trifluoro-4,6-dinitro-o- | (trifluoromethyl)-1,3- |
| | toluidine | benzenediamine |
| | or | |
| | 2,6-dinitro-N1,N1-dipropyl- | |
| | 4-trifluoromethyl-m- | |
| | phenylenediamine | |
| profluralin | N-cyclopropylmethyl-α,α,α- | N-(cyclopropylmethyl)-2,6- |
| | trifluoro-2,6-dinitro-N- | dinitro-N-propyl-4- |
| | propyl-p-toluidine | (trifluoromethyl)benzenamine |
| | or | |
| | N-cyclopropylmethyl-2,6- | |
| | dinitro-N-propyl-4- | |
| | trifluoromethylaniline | |
| trifluralin (TREFLAN, | α,α,α-trifluoro-2,6-dinitro- | 2,6-dinitro-N,N-dipropyl-4- |
| TRIFIC, TRILLIN) | N,N-dipropyl-p-toluidine | (trifluoromethyl)benzenamine |

Phosphoroamidates

| | | |
|---|---|---|
| AMP (Amiprofos | | |
| methyl); amiprophos- | | |
| methyl | | |
| Butamifos | O-ethyl O-6-nitro-m-tolyl | O-ethyl O-(5-methyl-2- |
| | (RS)-sec- | nitrophenyl) (1- |
| | butylphosphoramidothioate | methylpropyl)phosphoramidothioate |

Pyridines

| | | |
|---|---|---|
| Dithiopyr | | |
| Thiazopyr | methyl 2-difluoromethyl-5- | methyl 2-(difluoromethyl)-5- |
| | (4,5-dihydro-1,3-thiazol-2- | (4,5-dihydro-2-thiazolyl)-4-(2- |
| | yl)-4-isobutyl-6- | methylpropyl)-6- |
| | trifluoromethylnicotinate | (trifluoromethyl)-3- |
| | | pyridinecarboxylate |

The chromosome doubling agent may come in contact with the embryo at various times. If the embryo is isolated the doubling agent may come in contact immediately after isolation and before germination. If the embryo is contained within the seed, it may come in contact with the doubling agent any time after pollination and before dry-down. The embryo whether it is isolated or not may come in contact with the doubling agent any time between 6 hours after pollination and 21 days after pollination. The duration of contact between the chromosomal doubling agent may vary. Contact may be from less than 24 hours, for example 4-12 hours, to about a week. The duration of contact is generally from about 24 hours to 2 days.

Methods provided may or may not go through a callus formation stage. The haploid embryos may be placed on a “non-callus promoting medium. The term “non-callus promoting medium” refers to a medium that does not support proliferation of dedifferentiated masses of cells or tissue. A preferred “non-callus promoting medium” is used for embryo rescue, containing typical salt and vitamin formulations well known in the art. Such embryo rescue, or embryo culture, media contain little or no auxin [for review see Raghaven, V., 1966. Biol. Rev. 41:1-58]. Embryo maturation medium also represents another preferred “non-callus promoting medium”. Embryo maturation medium is used to promote development of in vitro cultured embryos, preventing precocious germination, and typically contain standard salt/vitamin formulations increased sugar levels and/or exogenously added abscisic acid, with little or no auxin. Another type of medium is used for shoot culture, or multiple shoot proliferation. This multiple-shoot medium can again contain little or reduced auxin, but instead contain elevated levels of cytokinin that promote meristem proliferation and growth.

An auxin is defined as an endogenous plant hormone such as indole acetic acid (IAA), derivatives of IAA such as indole 3 buteric acid, as well as compounds with auxin-like activity such as 2,4-D, picloram, dicamba, 3,4-D, 2,4,5-T and naphthalene acetic acid (NAA).

A cytokinin is defined as a naturally occurring plant hormone such as 2-isopentynel adenine (2iP), zeatin and hidydrozeatin, or a synthetic compound with cytokinin-like activity such as kinetin and BAP (beynzylaminopurine).

Polynucleotides or polypeptides involved in growth stimulation or cell cycle stimulation can be used to increase the frequency of haploid embryos produced, increase the recovery of haploid plants, and/or stimulate chromosomal doubling efficiency. The growth stimulation polynucleotide can be provided by the female parent. The growth stimulation polynucleotide or polypeptide can be provided by stable or transient transformation. Polynucleotides whose overexpression has been shown to stimulate the cell cycle include Cyclin A, Cyclin B, Cyclin C, Cyclin D, Cyclin E, Cyclin F, Cyclin G, and Cyclin H; Pinl; E2F; Cdc25; RepA and similar plant viral polynucleotides encoding replication associated proteins. See U.S. Pat. No. 8,865,971.

After successful doubling of the haploid chromosomes, it may be desirable to remove the above growth stimulation polynucleotides. This can be accomplished by using various methods of gene excision, such as with the use of recombination sites and recombinases.

Haploid cells from embryos, seeds, plants, etc. can be identified by several methods, such as, by chromosomal counts or by use of a flow cytometer. Flow cytometers are available from several vendors including, but not limited to BD Biosciences, 2350 Qume Drive, San Jose, Calif. 95131-1807, USA, for example, Model # BD Accuri™ C6 Plus. Flow cytometer tests are performed using a standard flow cytometer test protocol in accordance with the manufacturer's instructions.

Molecular markers or quantitative PCR can be used to determine if a tissue or plant is made of doubled haploid cells or is made of diploid cells (cells obtained through normal pollination or somatic tissue from the heterozygous parent).

Transformation of the haploid embryo may also be used in the methods. The type of transformation is not critical to the methods; various methods of transformation are currently available. As newer methods are available to transform host cells they may be directly applied. Accordingly, a wide variety of methods have been developed to insert a DNA sequence into the genome of a host cell to obtain the transcription and/or translation of the sequence. Thus, any method that provides for efficient transformation/transfection may be employed.

Methods for transforming various host cells are disclosed in Klein et al. “Transformation of microbes, plants and animals by particle bombardment”, Bio/Technol. New York, N.Y, Nature Publishing Company, March 1992, 10(3):286-291. Techniques for transforming a wide variety of higher plant species are well known and described in the technical, scientific, and patent literature. See, for example, Weising et al, Ann. Rev. Genet. 22:421-477 (1988). For example, the DNA construct may be introduced directly into the genomic DNA of the plant cell using techniques such as electroporation, PEG-induced transfection, particle bombardment, silicon fiber delivery, or microinjection of plant cell protoplasts or embryogenic callus. See, e.g. Tomes et al. Direct DNA Transfer into Intact Plant Cells Via Microprojectile Bombardment, pp. 197-213 in Plant Cell, Tissue and Organ Culture, Fundamental Methods, eds. 0. L. Gamborg and G. C. Phillips. Springer-Verlag Berlin Heidelberg New York, 1995. The introduction of DNA constructs using polyethylene glycol precipitation is described in Paszkowski et al, Embo J. 3:2717-2722 (1984). Electroporation techniques are described in Fromm et al. Proc. Natl. Acad. Sci. 82:5824 (1985). Ballistic transformation techniques are described in Klein et al. Nature 327:70-73 (1987).

Alternatively, the DNA constructs may be combined with suitable T-DNA flanking regions and introduced into an Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-meditated transformation techniques are well described in the scientific literature. See, for example Horsch et al. Science 233:496-498 (1984), and Fraley et al. Proc. Natl. Acad. Sci. 80: 4803 (1983). For instance, Agrobacterium transformation of maize is described in U.S. Pat. No. 5,981,840. Agrobacterium transformation of monocot is found in U.S. Pat. No. 5,591,616. Agrobacterium transformation of soybeans is described in U.S. Pat. No. 5,563,055.

Other methods of transformation include (1) Agrobacterium rhizogenes-induced transformation (see, e.g, Lichtenstein and Fuller In: Genetic Engineering, vol. 6, P W J Rigby, Ed, London, Academic Press, 1987; and Lichtenstein, C. P, and Draper, J. In: DNA Cloning, Vol. 11, D. M. Glover, Ed, Oxford, IRI Press, 1985), Application PCT/US87/02512 (WO 88/02405 published Apr. 7, 1988) describes the use of FA. rhizogenes strain A4 and its Ri plasmid along with A. tumefaciens vectors pARC8 or pARC16 (2) liposome-induced DNA uptake (see, e.g. Freeman et al. Plant Cell Physiol. 25:1353, 1984), (3) the vortexing method (see, e.g. Kindle, Proc. Natl. Acad. Sci, USA 87:1228, (1990), (4) Ochrobactrum-mediated transformation (see U.S. Pat. Pub. No. US20180216123A1). (5) Rhizobiaceae-mediated transformation bacteria (see U.S. Pat. No. 9,365,859), and (6) rapid plant transformation (see U.S. Pat. Pub. No. US20170121722A1).

Transformed haploid embryos which are derived by any of the above transformation techniques can be cultured to regenerate a whole plant which possesses the transformed genotype. Such regeneration techniques may be considered as embryo rescue.

Embryo rescue media can comprise certain phytohormones and energy sources or just energy sources. The growth medium may also contain a selection agent such as a biocide and/or herbicide. This selection agent can be used to indicate a marker which has been introduced through the transformation process.

Generation of embryos into plants is well known in the art. Embryo rescue techniques can be used to generate immature doubled haploid embryos into plants (Recent Research Developments in Genetics & Breeding. Vol. 1, Part II, 287-308 2004). The disclosure of which is herein incorporated by reference.

The temperature at which the methods can be performed can vary. The methods provided can be practiced at any temperature that does not kill a plant cell or plant or from about 16 degrees Celsius to 32 degrees Celsius. Any or all or any combination of the various steps of the invention: embryo isolation, culturing, embryo cell doubling may be performed in the light or dark.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only, and are not intended to limit the scope of the disclosure in any way. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims. Efforts have been made to ensure accuracy with respect to numbers used (e.g., amounts, temperatures, etc.), but some experimental error and deviation should, of course, be allowed for.

### Example 1: Testing Different Species Pollen Donors for Sorghum Haploid Production

A number of genetically distant species from sorghum i.e., maize, wheat, pearl millet, canola, cotton, barley, rice, cogon grass, eastern gamma grass, teosinte, and sunflower were each used as a pollen donor and were each crossed with female sorghum diploid plants for wide hybridization crosses. These species exhibited varying haploid frequencies. Pearl millet, which has a smaller pollen grain size and produces abundant pollen grains generated significantly increased sorghum haploids compared to the other pollen donor species listed above.

### Example 2: Producing a Population of Haploid Sorghum Plants

Seeds from F1 or F2 or F3 sorghum plants were planted and the resulting plants were used as female parent plants (pollen receivers). Alternatively, any backcross derived sorghum plant can be planted and the resulting plants are used as female parent plants (pollen receivers). Seeds from pearl millet (Pennisetum glaucum) (2n=14), for example pearl millet inbred line PHI01A, were planted and the resulting plants were used as male parent plants (pollen donors). This was an interspecific crossing between sorghum and pearl millet. The anthers of the pollen receiver sorghum lines were removed by hand or application of a hot water treatment to produce emasculated sorghum spikelets. The stigmas on the sorghum female parent plants were pollinated (fertilized) with viable pollen grains collected from the anthers of the pearl millet male parent plants (used as haploid inducer plants). This was a controlled pollination, using a paint brush under a controlled environment to ensure the female stigma received pollen grains. After pollination for 24 hours, a plant growth regulator 2,4-D (2,4-Dichlorophenoxyacetic acid) solution was used to spray spikelets pollinated with pearl millet pollen. Twenty-four hours after pollination, a 50-200 mg/L solution of the plant growth regulator 2,4-D (2,4-Dichlorophenoxyacetic acid) was sprayed on the pollinated spikelets and these spikelets were allowed to continue their growth on the female sorghum parent plant. This pollination method resulted in the production of haploid embryos in each sorghum head (inflorescence) at a frequency of about 2-5%. The pearl millet chromosomes were eliminated at mitosis during an early embryo cell division stage after fertilization. At approximately 10-20 days after pollination the heads were harvested. Alternatively, the heads are harvested at approximately 14-17 days after pollination. The heads were surface sterilized in 70% bleach for 1 minute, and rinsed two times with sterile water, then submersed again in 60% bleach for 10 minutes and then rinsed 3-5 times with sterile water. The haploid embryos were rescued from the heads. For example, out of 100 caryopsis excised under the microscope using a scalpel and forceps, only 2-4 contained haploid embryos identified as floating inside the caryopsis, that was filled with water, but had no endosperm.

This interspecific crossing produced maternal haploids having only one set of chromosomes from the female parent (sorghum) in the embryo cells. After the haploid sorghum embryos were rescued as described above, they were placed on MS germination media and incubated in the dark. After 2-3 weeks of growing in the dark, the rescued embryos were transferred onto fresh media and placed in a light culture room to grow into plantlets. Approximately 2 weeks later, seedlings were transferred into tubes containing nutrient rich media and grown for 1 week in a light culture room and subsequently moved to the greenhouse for growth to maturity. Leaf samples from seedlings were collected and underwent a flow cytometer test in accordance with the manufacturer's instructions, for example, BD Biosciences, Flow Cytometer, Model# BD Accuri™ C6 Plus, 2350 Qume Drive San Jose, Calif. 95131-1807, USA, to confirm their ploidy level. Results are shown in Table 2.

This method resulted in generation of a heterogeneous population of haploid sorghum plants. These haploid sorghum plants were phenotypically male sterile and can be further evaluated for breeding purposes. Nine additional pearl millet lines i.e., PI 287023, PI 213011, PI 295124, PI 296377, PI 331692, PI 595755, PI 185642, PI 186338, and PI 634535 were also used as pollen donors for haploid induction and provided results similar to those shown in Table 2 below. As shown in Table 2, multiple diploid sorghum genotypes were crossed with pearl millet line PHI01A. In addition, some experiments were repeated in multiple years, as shown in Table 2.

TABLE 2

| | Embryo Age | | | | | | | | |
|---|---|---|---|---|---|---|---|---|---|
| | (Days After | | | | | | | | |
| | Pollination | | | | | | Number | | |
| Diploid | With Pearl | Number Of | Number Of | Percent | | Percent | Flow | Number Of | Percent |
| Sorghum | Millet | Caryopsis | Embryos | Embryo | Number Of | Plantlet | Cytometer | Haploids | Haploid |
| Genotype/Year | Pollen) | Excised | Recovered | Induction | Plantlets | Germination | Tested | Recovered | Induction |
| PHI01/2018 | 14 | 38,310 | 932 | 2.4% | 143 | 15.3% | 110 | 15 | 14% |
| PHI02/2018 | 14 | 101,403 | 2,261 | 2.2% | 706 | 31.2% | 606 | 63 | 10% |
| PHI02/2019 | 14 | 33,687 | 782 | 2.3% | 210 | 26.9% | 180 | 17 | 9% |
| PHI02/2020 | 14 | 18,145 | 1064 | 5.9% | 163 | 15.3% | 145 | 13 | 9% |
| PHI03/2018 | 14 | 41,432 | 753 | 1.8% | 188 | 25.0% | 136 | 20 | 15% |
| PHI04/2018 | 14 | 4,726 | 26 | 0.6% | 11 | 42.3% | 9 | 9 | 100% |
| PHI05/2018 | 14 | 3,069 | 15 | 0.5% | 7 | 46.7% | 5 | 3 | 60% |
| PHI05/2019 | 14 | 14,831 | 40 | 0.3% | 5 | 12.5% | 4 | 4 | 100% |
| PHI05/2020 | 14 | 10,086 | 8 | 0.1% | 1 | 12.5% | 1 | 1 | 100% |
| PHI06/2018 | 14 | 2,476 | 59 | 2.4% | 28 | 47.5% | 25 | 24 | 96% |
| PHI07/2018 | 14 | 2,935 | 85 | 2.9% | 26 | 30.6% | 24 | 24 | 100% |
| PHI08/2018 | 14 | 2,030 | 15 | 0.7% | 5 | 33.3% | 2 | 2 | 100% |
| PHI09/2018 | 14 | 31,784 | 298 | 0.9% | 73 | 24.5% | 57 | 8 | 14% |
| PHI09/2019 | 14 | 28,923 | 659 | 2.3% | 220 | 33.4% | 176 | 14 | 8% |
| PHI09/2020 | 14 | 11,134 | 398 | 3.6% | 89 | 22.4% | 80 | 10 | 13% |
| PHI10/2018 | 14 | 26,104 | 297 | 1.1% | 127 | 42.8% | 98 | 38 | 39% |
| PHI10/2019 | 14 | 22,589 | 441 | 2.0% | 217 | 49.2% | 193 | 36 | 19% |
| PHI10/2020 | 14 | 13,631 | 404 | 3.0% | 180 | 44.6% | 166 | 25 | 15% |
| PHI11/2019 | 14 | 36,463 | 429 | 1.2% | 67 | 15.6% | 47 | 2 | 4% |
| PHI11/2020 | 14 | 11,501 | 269 | 2.3% | 32 | 11.9% | 32 | 2 | 6% |
| PHI12/2019 | 14 | 44,203 | 237 | 0.5% | 32 | 13.5% | 24 | 8 | 33% |
| PHI12/2020 | 14 | 19,391 | 190 | 1.0% | 42 | 22.1% | 38 | 1 | 3% |
| PHI13/2019 | 14 | 41,892 | 1050 | 2.5% | 168 | 16.0% | 122 | 30 | 25% |
| PHI13/2020 | 14 | 12,942 | 745 | 5.8% | 88 | 11.8% | 71 | 18 | 25% |
| PHI14/2019 | 14 | 59,750 | 1817 | 3.0% | 485 | 26.7% | 352 | 53 | 15% |
| PHI14/2020 | 14 | 21,365 | 832 | 3.9% | 150 | 18.0% | 116 | 14 | 12% |
| PHI15/2019 | 14 | 56,260 | 645 | 1.1% | 188 | 29.1% | 149 | 15 | 10% |
| PHI15/2020 | 14 | 12,504 | 325 | 2.6% | 90 | 27.7% | 80 | 5 | 6% |
| PHI16/2019 | 14 | 30,340 | 345 | 1.1% | 205 | 59.4% | 176 | 27 | 15% |
| PHI17/2020 | 14 | 13,199 | 344 | 2.6% | 92 | 26.7% | 77 | 4 | 5% |
| PHI18/2020 | 14 | 9,999 | 229 | 2.3% | 84 | 36.7% | 60 | 6 | 10% |
| PHI19/2020 | 14 | 14,029 | 373 | 2.7% | 177 | 47.5% | 139 | 23 | 17% |
| PHI20/2020 | 14 | 10,760 | 254 | 2.4% | 140 | 55.1% | 122 | 9 | 7% |
| Total | | 801,893 | 16,621 | 2.1% | 4439 | 26.7% | 3622 | 543 | 15% |

### Example 3: Producing a Double Haploid Population of Sorghum Plants

Chromosome doubling of the haploid sorghum plants generated in Example 2 can be accomplished using any doubling agent and any means known in the art such as for example, colchicine, pronamide, dithipyr, oryzalin, AMP, and trifluralin, or any of the other chromosome doubling agents referenced in Table 1. Specifically, chromosome doubling is accomplished through root soaking of young haploid seedlings where roots are exposed to doubling agents for a given period, typically from 2 hours to 12 hours. The seedling roots uptake the doubling agent and affect the mitotically multiplying meristem cells. Chromosome doubling occurs in these mitotically dividing cells when a single mitotically dividing single cell forms multiple cells. Prior to chromosome doubling meristematic tissues have differentiated into male and female gametophytes. As a result of doubling treatment, the chromosome number of haploid cells are doubled from n=10 to 2n=20. During development the plants transitions from vegetative to reproductive stage. If doubling occurs, the doubled sectors also transition into reproductive stage and produce partial to fully fertile plants. The developed doubled seedlings are then transplanted into pots under controlled conditions or in the field to establish their normal growth. These putative DH plants will be fertile, allowing recovery of homozygous inbred D1 seed.

### Example 4: Obtaining Double Haploid Sorghum Plants

Plants from diploid sorghum genotypes, “PHI01”, “PHI02”, “PHI03”, “PHI04”, “PHI05”, “PHI06”, “PHI07”, “PHI08”, “PHI09”, “PHI10”, “PHI11”, “PHI12”, “PHI13”, “PHI14”, “PHI15”, “PHI16”, “PHI17”, “PHI18”, “PHI19”, and “PHI20” were pollinated with pollen obtained from pearl millet line PHI01A, as described in Example 2. Immature haploid embryos were isolated, as described in Example 2, 10-20 days after pollination and embryo sizes ranged from 0.1-3 mm long. These haploid embryos were germinated on a medium (see Table 4) in the dark. The germinated haploid embryos were then transferred to fresh media for an additional 7-10 days at 21°−26° C. in the dark until germinated plantlets formed. The germinated plantlets were then transferred to a light culture room and grown at 21°−26° C. for another 2-3 weeks until healthy plantlets having good roots developed. These plantlets were then potted into soil and grown in a regular greenhouse for 5-6 weeks. Upon healthy plant establishment, the roots were treated with a chromosome doubling agent, colchicine, for 5 hours in the dark. Additional chromosome doubling agents for example, pronamide, dithipyr, oryzalin, AMP, and trifluralin, or any of the other chromosome doubling agents referenced in Table 1 can also be used. After treatment with the doubling agent, the roots were then thoroughly rinsed with running water for 30 min, to remove the residual doubling agent and then potted in soil and grown to maturity. The results are shown in Table 3. In addition, some experiments were repeated in multiple years, as shown in Table 3.

TABLE 3
Results

| | Time | | | Number | Number | |
|---|---|---|---|---|---|---|
| | duration of | | Number | of plants | of Fully | DH |
| Diploid | doubling | Colchicine | of haploid | survived | Fertile | Success |
| Sorghum | treatment | concentration | plants | after | DH | rate |
| Genotype/Year | (hours) | (%) | treated | treatment | plants | (%) |
| PHI01/2018 | 5 | 0.05 | 15 | 15 | 15 | 100% |
| PHI02/2018 | 5 | 0.05 | 63 | 62 | 62 | 98% |
| PHI02/2019 | 5 | 0.05 | 17 | 17 | 17 | 100% |
| PHI02/2020 | 5 | 0.05 | 13 | 13 | 13 | 100% |
| PHI03/2018 | 5 | 0.05 | 20 | 20 | 20 | 100% |
| PHI04/2018 | 5 | 0.05 | 9 | 9 | 9 | 100% |
| PHI05/2018 | 5 | 0.05 | 3 | 3 | 3 | 100% |
| PHI05/2019 | 5 | 0.05 | 4 | 4 | 4 | 100% |
| PHI05/2020 | 5 | 0.05 | 1 | 1 | 1 | 100% |
| PHI06/2018 | 5 | 0.05 | 24 | 24 | 24 | 100% |
| PHI07/2018 | 5 | 0.05 | 24 | 23 | 23 | 96% |
| PHI08/2018 | 5 | 0.05 | 2 | 2 | 2 | 100% |
| PHI09/2018 | 5 | 0.05 | 8 | 8 | 8 | 100% |
| PHI09/2019 | 5 | 0.05 | 14 | 14 | 13 | 93% |
| PHI09/2020 | 5 | 0.05 | 10 | 10 | 10 | 100% |
| PHI10/2018 | 5 | 0.05 | 38 | 38 | 38 | 100% |
| PHI10/2019 | 5 | 0.05 | 36 | 35 | 35 | 97% |
| PHI10/2020 | 5 | 0.05 | 25 | 25 | 25 | 100% |
| PHI11/2019 | 5 | 0.05 | 2 | 2 | 2 | 100% |
| PHI11/2020 | 5 | 0.05 | 2 | 2 | 2 | 100% |
| PHI12/2019 | 5 | 0.05 | 8 | 8 | 8 | 100% |
| PHI12/2020 | 5 | 0.05 | 1 | 1 | 1 | 100% |
| PHI13/2019 | 5 | 0.05 | 30 | 30 | 30 | 100% |
| PHI13/2020 | 5 | 0.05 | 18 | 18 | 18 | 100% |
| PHI14/2019 | 5 | 0.05 | 53 | 52 | 52 | 98% |
| PHI14/2020 | 5 | 0.05 | 14 | 13 | 13 | 93% |
| PHI15/2019 | 5 | 0.05 | 15 | 15 | 15 | 100% |
| PHI15/2020 | 5 | 0.05 | 5 | 5 | 5 | 100% |
| PHI16/2019 | 5 | 0.05 | 27 | 27 | 27 | 100% |
| PHI17/2020 | 5 | 0.05 | 4 | 4 | 4 | 100% |
| PHI18/2020 | 5 | 0.05 | 6 | 6 | 6 | 100% |
| PHI19/2020 | 5 | 0.05 | 23 | 23 | 23 | 100% |
| PHI20/2020 | 5 | 0.05 | 9 | 9 | 9 | 100% |
| Sum | | | 543 | 538 | 537 | 99% |

TABLE 4
Media

| Medium components | Units per liter | 13292 |
|---|---|---|
| MS BASAL SALT MIXTURE | g/L | 2.165 |
| SUCROSE | g/L | 30.0 |
| GELZAN | g/L | 4.0 |
| 13010-CEFOTAXIME 250 MG/ML | mg/L | 0.25 |
| (13010BASE VAX 17A) | | |
| pH | | 5.7 |

### Example 5: Haploid Induction and Genome Editing of Sorghum Through Wide-Cross/Out-Cross Hybridization

Haploid induction and genome editing in sorghum is accomplished by using pearl millet pollen as the editing donor line in a wide-cross out-cross for sorghum. In sorghum, haploid induction is performed by using a pollen donor from a different species (e.g., pearl millet) that can trigger induction but does not contribute to the genetic composition of the resulting haploid embryo, but the wide-cross delivers one or more components of the genome editing machinery to make targeted edits in a haploid cell. For example, in a gynogenetic haploid induction it is desirable for the male donor line to contain the genome editing machinery, because the pollen-derived DNA (chromosomal segments) is eliminated in the haploid induction process. Similarly, for androgenic haploid induction, the genome editing machinery can be present in the female parent, because the female chromosomes are eliminated in the haploid induction process.

Transformable pearl millet lines are selected and a stable pearl millet line expressing one or more of the genome editing machinery components (e.g., Cas9, cpf1, base editing deaminases, guide RNAs, zinc finger nucleases, and other site-specific nucleases/enzymes) is selected for contemporaneous genome editing plus haploid induction. These pearl millet lines can either have the one or more genome editing machinery components stably integrated and expressing or transiently present in the pollen to deliver during the induction process. The line that receives the edits could be elite sorghum germplasm, and the editing machinery is generally eliminated during the haploid induction process. Edited doubled haploid lines are produced without direct transformation of sorghum lines and culturing.

Transgenic genome editing locus is introduced into pearl millet lines used for wide-crosses to induce haploid induction and targeted genome editing of sorghum lines. In an embodiment, the transgenic locus is made homozygous and then the pearl millet line is used as a pollen donor in a wide-cross with recipient sorghum to induce haploids to produce desired edits. Post-fertilization genome elimination in wide-crosses is carried out where the pearl millet genome or a genetic component thereof is eliminated during cell division/embryo development resulting in double haploid embryos devoid of interspecific DNA. In an embodiment, a promoter that drives expression of one or more components of the genome editing machinery in the pollen, sperm cells, or a zygote cell or a combination thereof is used to enable the editing guide RNA and protein to be present at the destination where the genome editing takes place.

In an embodiment, contemporaneous haploid induction and editing via wide-cross with pearl millet as a pollen donor (carrying one or more components of the genome editing machinery) and sorghum as the pollen recipient is demonstrated by providing a transgenic pearl millet line expressing Cas9 and sgRNA targeting a polynucleotide of interest in the sorghum genome. Pollen collected from transgenic pearl millet TO or progeny T1 plants carrying the genome editing machinery are used to pollinate emasculated sorghum lines as described in the Examples above. Embryos are extracted from pollinated sorghum spikelets as described in Example 4 and further grown to obtain edited doubled haploid plants.

### Example 6: Haploid Induction and Generation of Doubled Haploid Sorghum Plants from Early Embryo or Embryo-Like Structures

Haploid induction and genome editing in sorghum is accomplished by using a haploid inducer line of sorghum or through a wide-cross, such as for example using pearl millet pollen as a donor line. Approximately at about 2-30 days, preferably about 4-20 or about 10-15 days after pollination/induction, the sorghum heads are harvested. The heads are surface sterilized for example, using sterilization techniques such as soaking in 70% bleach for 1 minute, and rinsing two times with sterile water, then submersed again in 60% bleach for 10 minutes and then rinsed 3-5 times with sterile water. The haploid embryos are rescued from the heads. Haploid induction produces maternal haploids having only one set of chromosomes from the female parent (sorghum) in the embryo cells. After the haploid sorghum embryos are rescued, they are placed on a germination media containing a chromosome doubling agent and incubated in the dark. After sufficient time, e.g., 4 hours to about 48 hours, the rescued sorghum embryos are transferred to a growth media not containing any doubling agent. The doubling agent treatment and initial germination of the rescued embryos may be done in dark, under suitable growth conditions. The growth media may be suitable for germination of embryo cells into plantlets without substantial formation of callus. About 2-3 weeks of growing in the dark, the rescued embryos are transferred onto fresh media and placed in a light culture room to grow into plantlets and subsequently into soil for further analysis including seed set.

### Example 7: Automated, High-Throughput Generation of Doubled Haploid Sorghum Plants for Production in a Sorghum Breeding Program

Haploid induction and genome editing in sorghum is accomplished by using a haploid inducer line of sorghum or through a wide-cross, such as for example using pearl millet pollen as a donor line. Wide-cross pollination or pollination using an inducer sorghum pollen donor plant is accomplished in the field or in a greenhouse using one or more automated or semi-automated mechanized pollen applicators. Approximately at about 2-30 days, preferably about 4-20 or about 10-15 days after pollination/induction, the sorghum heads are harvested in a high-throughput manner, for example, by utilizing an automated sorghum head harvestor by a combine. The heads are surface sterilized for example, using sterilization techniques such as soaking in 70% bleach for 1 minute, and rinsing two times with sterile water, then submersed again in 60% bleach for 10 minutes and then rinsed 3-5 times with sterile water, wherein one or more of these steps are automated.

Supply of immature embryos from haploid induced sorghum plants is accomplished by automated/semi-automated mechanical means. Conditions of flowering are monitored and harvesting window for harvesting the heads after pollination are predicted using sorghum crop growth models. This can be accomplished by deep learning/machine learning methods that take into account, the weather, genotype, haploid induction timing and other factors that help obtain immature sorghum haploid embryos. Manual embryo excision from harvested panicles, while possible, is assisted with one or more automation steps and in certain embodiments, removal of the mature or immature embryos from sorghum seeds/heads are fully automated. Optionally, following haploid induction by a sorghum male haploid inducer, the sorghum haploid embryos are identified by the expression of a phenotypic marker—e.g., pigmentation or lack thereof (in the case of a haploid). Alternatively, genotypic selection is also accomplished based on for example, non-destructive or semi-destructive RNA profiling.

The sorghum embryos are separated from the heads using a mechanical separator. After the haploid sorghum embryos are rescued, they are placed on a germination media containing a chromosome doubling agent and incubated in the dark. After sufficient time, e.g., 4 hours to about 48 hours, the rescued sorghum embryos are transferred to a growth media not containing any doubling agent. The doubling agent treatment and initial germination of the rescued embryos may be done in dark, under suitable growth conditions. The growth media may be suitable for germination of embryo cells into plantlets without substantial formation of callus. About 2-3 weeks of growing in the dark, the rescued embryos are transferred onto fresh media and placed in a light culture room to grow into plantlets and subsequently into soil for further analysis including seed set.

Double haploid sorghum plants are then used in a breeding program to produce hybrid sorghum plants. The methods described herein are also adaptable for engineering genome modification to a haploid sorghum embryo. Similarly, transgenes can also be introduced at the haploid embryo stage, e.g., for direct transformation of elite sorghum lines, including sorghum lines that are generally considered as recalcitrant for genetic transformation techniques.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

1. A method of producing a sorghum haploid embryo, the method comprising pollinating a female sorghum diploid plant with pollen from a Pennisetum plant and obtaining the sorghum haploid embryo.

2.-3. (canceled)

4. The method of claim 1, wherein the sorghum plant is emasculated.

5. The method of claim 1, wherein the Pennisetum plant is selected from the group consisting of Pennisetum glaucum, Pennisetum cenchroides, Pennisetum americanum (L.), Pennisetum typhoides auct., Cenchrus ciliaris L, and Panicum sumatrense.

6.-8. (canceled)

9. The method of claim 1, further comprising generating a sorghum haploid seedling from the sorghum haploid embryo.

10.-12. (canceled)

13. The method of claim 1, further comprising exposing the sorghum haploid embryo to a chromosome doubling agent.

14. (canceled)

15.-29. (canceled)

30. The method of claim 13, wherein the chromosome doubling agent is selected from the group consisting of colchicine, pronamide, dithipyr, oryzalin, AMP, and trifluralin.

31.-33. (canceled)

34. A method of producing a sorghum doubled haploid embryo, the method comprising contacting a sorghum female reproductive system or a component thereof with a pearl millet or a genetically related species of pearl millet male reproductive system or a component thereof to produce a haploid sorghum cell; contacting the haploid sorghum cell with a chromosome doubling agent; and producing the sorghum doubled haploid embryo from the haploid sorghum cell.

35. The method of claim 34, further comprising growing the sorghum doubled haploid embryo to a sorghum doubled haploid plant.

36.-37. (canceled)

38. The method of claim 34, wherein the pearl millet or the genetically related species of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens.

39. The method of claim 34, wherein the chromosome doubling agent is selected from the group consisting of colchicine, pronamide, dithipyr, oryzalin, AMP, and trifluralin.

40. A method of obtaining a doubled haploid sorghum plant, the method comprising:
(a) pollinating an emasculated sorghum diploid spikelet with pollen of a diploid pearl millet plant or a diploid plant of a close genetic relative of pearl millet to produce at least one haploid sorghum embryo or at least one haploid sorghum seedling;
(b) applying a growth regulator to the at least one haploid sorghum embryo or the at least one haploid sorghum seedling;
(c) isolating the at least one haploid sorghum embryo from caryopsis between 10-20 days after step (a);
(d) obtaining the at least one haploid sorghum seedling between 3-4 weeks after step (b);
(e) confirming the ploidy level of the isolated haploid sorghum embryo of (c) or the isolated haploid sorghum seedling of (d);
(f) contacting the haploid sorghum embryo of (c) or the haploid sorghum seedling of (d) with a chromosome doubling agent to produce at least one doubled haploid sorghum embryo cell or at least one doubled haploid sorghum seedling cell;
(g) culturing the doubled sorghum embryo cell or the doubled sorghum seedling cell on a germination medium; and
(h) generating a doubled haploid sorghum plant from the doubled haploid sorghum embryo cell or the doubled haploid sorghum seedling cell.

41. The method of claim 40, wherein the emasculated sorghum diploid spikelet is an anther emasculated sorghum diploid spikelet.

42. The method of claim 40, wherein the haploid sorghum embryo of step (b) is 0.1 mm to 3 mm in length.

43. The method of claim 42, wherein the at least one haploid sorghum embryo of step (c) is isolated from caryopsis between 14-17 days after step (a).

44. (canceled)

45. The method of claim 40, wherein the chromosome doubling agent is selected from the group consisting of colchicine, pronamide, dithipyr, oryzalin, AMP, and trifluralin.

46. The method of claim 40, wherein in step (f) the haploid sorghum embryo or haploid sorghum seedling is in contact with the chromosome doubling agent for 3 hours to 48 hours.

47. The method of claim 40, wherein the diploid pearl millet plant or the diploid plant of a close genetic relative of pearl millet is selected from the group consisting of Pennisetum glaucum, Pennisetum cenchroides, Pennisetum americanum (L.), Pennisetum typhoides auct., Cenchrus ciliaris L, Panicum sumatrense, Panicum americanum L, Setaria viridis, Eleusine coracana, Cenchrus americanus (L.), Setaria glauca (L.), and Setaria lutescens.

48.-75. (canceled)
